(19) United States
(12) Patent Application Publication
(10) Pub. No.: US 2020/0360685 A1
(43) Pub. Date: Nov. 19, 2020

(54) FLEXIBLE SINGLE-SIDED CONDUCTIVE MICROSTRUCTURE ARTIFICIAL COCHLEA ELECTRODE AND PRODUCTION METHOD

(71) Applicant: SHANDONG UNIVERSITY, Jinan (CN)

(72) Inventors: Lan TIAN, JINAN (CN); Xiaoshan LU, JINAN (CN); Shuzhong BAI, JINAN (CN)

(21) Appl. No.: 16/987,500

(22) Filed: Aug. 7, 2020

Related U.S. Application Data
(63) Continuation of application No. PCT/CN2018/125450, filed on Dec. 29, 2018.

(30) Foreign Application Priority Data
Feb. 13, 2018 (CN) 201810148399.6

(51) Int. Cl. A61N 1/05; A61N 1/36; H04R 25/00; H01B 3/46

(52) U.S. Cl. CPC A61N 1/0541; A61N 1/36038; H04R 2225/67; H01B 3/46; H04R 25/658

(57) ABSTRACT

Disclosed is a flexible single-sided conductive microstructure artificial cochlea electrode. The artificial cochlea electrode comprises a flexible biocompatible insulation material layer. An upper layer of the flexible biocompatible insulation material layer comprises a conductive metal layer and an adhesion layer. The conductive metal layer comprises an electrode area, a lead area and a pin area. A plurality of leads are etched in the lead area. A plurality of electrodes are etched in the electrode area. One electrode is connected with one lead; and the pin area is provided with pins corresponding to the leads of the lead area one by one.

### CROSS-REFERENCE TO RELATED APPLICATIONS

This application is a continuation of International Patent Application No. PCT/CN2018/125450 with a filing date of Dec. 29, 2018, designating the United States, now pending, and further claims priority to Chinese Patent Application No. 201810148399.6 with a filing date of Feb. 13, 2018. The content of the aforementioned applications, including any intervening amendments thereto, are incorporated herein by reference.

### TECHNICAL FIELD

The present invention relates to a flexible artificial cochlea nerve stimulation electrode with a single-sided conductive film microstructure and a production method.

### BACKGROUND OF THE PRESENT INVENTION

A large number of nerve endings capable of receiving external electric stimulation are distributed inside cochlea according to positions. External sound signals can be collected, analyzed and coded to be connected with the nerve endings through the proper electric signal stimulation so as to activate auditory nerve conduction paths, which can be used for the treatment of disability of some auditory systems, such as an electronic cochlea or an artificial cochlea.

The interior of the cochlea is a curved structure and has narrow channels. There are other tissue structures in the channels, so that a sensing electrode for the auditory nerve stimulation should have good flexibility. For easy implanting, each exposed electrode end should be made into an annular shape (i.e. have no direction) as far as possible. Meanwhile, a flexible electrode array packaging material should have biocompatibility.

The shape, size, auditory nerve distribution and the like of the cochlea of individual patients may be different. The electrode structure is easy to design and produce, which is very important for precise and individual treatment.

On the premise of meeting the treatment requirement, more electrode contacts and a smaller overall aperture of a long conical electrode are also indispensable requirements for the precise treatment.

Patent 201610534724.3 discloses an artificial cochlea electrode based on liquid metal and a preparation method thereof, which has the following defects:

1. Due to local pressure during cochlear implantation or use, the electrode based on liquid metal may cause deformation of the liquid metal channel, affect the conductivity of the liquid metal, and even break in severe cases, which has hidden danger of unstable stimulation signal transmission.

2. If the electrode array based on the liquid metal has local electrode damage in the human body, the leakage of the liquid metal may occur, which causes secondary injury to the cochlea.

3. An exposed conductive area of the electrode array based on the liquid metal is in a point shape, but nerve cells receiving the electric stimulation are only distributed at one side of a cochlea axis in a tympanic tube, so that in order to make the implanted electrode play a role in stimulating the auditory nerves, the electrode conductive area must be faced to the cochlea axis. However, a tympanic cavity of the cochlea is a long tube coiled in two and a half circles, so that to make an electrode point of the implanted electrode face the cochlea axis, the operation requirements for doctors are excessively high and are difficult to meet.

### SUMMARY OF PRESENT INVENTION

To solve the technical problems proposed in the background, the present invention discloses a flexible artificial auditory nerve stimulation electrode and a production method.

The present invention adopts the following technical solutions:

A flexible single-sided conductive microstructure artificial cochlea electrode includes a flexible biocompatible insulation material layer. An upper layer of the flexible biocompatible insulation material layer is a conductive metal layer. The conductive metal layer includes an electrode area, a lead area and a pin area. A plurality of leads are etched in the lead area. A plurality of electrodes are etched in the electrode area. One electrode is connected with one lead. The pin area is provided with pins corresponding to the leads of the lead area one by one.

Further, the flexible single-sided conductive microstructure artificial cochlea electrode includes a flexible artificial auditory nerve stimulation electrode. Electrodes of the electrode area are exposed out of a packaged electrode array and contact auditory nerve cell tissues to transfer a stimulation electric signal; and the lead area is wrapped (or packaged) inside the flexible biocompatible insulation material layer.

Further, in a needle-shaped electrode area of the cochlea electrode, a diameter at the top cochleae surrounded by the electrodes may be 0.2 to 1.0 mm, and the diameter at the basis cochleae surrounded by the electrodes may be 1.0 to 5.0 mm.

A production method of the above flexible artificial auditory nerve stimulation electrode includes the following steps:

step 1, providing a hard substrate, and coating or paving the substrate with a flexible biocompatible insulation material layer;

step 2, spin coating the insulation material layer with photoresist, covering with a designed mask, and carrying out the exposure;

step 3, developing an exposed film in a developing solution, and heating at an appropriate temperature;

step 4, depositing or plating an adhesion layer at one side of the photoresist by using electron-beam evaporation;

step 5, depositing or plating a conductive metal layer at one side of the photoresist by using electron-beam evaporation;

step 6, washing the photoresist, or in order to protect a conductive structure, spin coating with another flexible insulation film, and solidifying, and stripping the solidified flexible biocompatible insulation material layer with the metal conductive structure from the hard substrate;

step 7, shaping; and

step 8, fixing and packaging.

Further, a shaping method in step 7 is as follows:

A long cylindrical or long conical filament is used as a support roller 4. The film is curled by adopting the support roller as an axis from a pin side away from the electrode area at a given angle or rolled from a film edge without a support into a needle shape, so that in this way, the pin area is exposed at the lower portion of the needle shape layer by layer; when the film is curled to a last circle, the electrode filament of the electrode area is curled at the upper portion of the outermost layer of a long conical tube in a direction approximately vertical to the support axis, thereby forming an approximately annular or U-shaped conductive electrode array. The leads curled in the lead area are separated by the insulation film material and insulated to one another.

Further, the process of step 8 is as follows: during the curling process, the film may be bonded while being curled, a suitable adhesive (such as liquid PDMS or other glue) may be smeared at the inner side of the prepared electrode array film until the outermost edge of the curled electrode area is adhered, followed by standing and fixation; and then the necessary electrode point-to-point electrical characteristic measurement and subsequent connection and package of the pins and electronic circuits of electrode array may be carried out.

Further, the flexible biocompatible insulation material layer in step 1 may be polydimethylsiloxane (PDMS) or other flexible biocompatible insulation materials.

According to the position distribution rule of the cochlea auditory nerve endings, the present invention utilizes the flexible and biocompatible film material to design and produce the flexible electric stimulation electrode array in steps of film preparation, metal deposition, curling and shaping, fixation and packaging, etc. The design and production method has the characteristics that the electrodes are firm, the electrodes can be densely distributed and can be customized, the overall aperture of the electrode array is small, and the like, thereby meeting the individual and precise treatment.

The present invention has the beneficial effects:

1. The present invention adopts single-layer or two-layer PDMS, so that the thickness of the electrode material is reduced, and the electrode array with small aperture is easy to prepare, thereby facilitating the number increase of the electrodes, and guaranteeing the flexibility.

2. The conductive material adopted by the present invention is solid metal, the solid metal with good ductility and high conductivity may be evaporated into the film, and the conductivity is relatively stable during the curling and use.

3. By using the solid metal, compared with the liquid metal, the present invention can avoid the possible secondary injury caused by the breaking leakage and is safer.

4. The electrode array produced by the present invention is easy for designing the annular or U-shaped electrode contacts, so that the conductive electrode is easy to align at and faced to the cochlear axis, and during the implanting operation, the electrode is convenient to operate and easy to face to the auditory nerves.

5. Moreover, the present invention is good in conductivity, optimal in flexibility, safe (adopting the biocompatible material), precise (the size is small and the electrode contacts are exquisite), reliable (the electrode is not likely to drop off), convenient to shape (curling the flexible film material), and simple in preparation process. The present invention is small in volume and high in flexibility, and can reduce the physical injury during the cochlea implanting process.

### DESCRIPTION OF THE DRAWINGS

The drawings of the description forming a part of the present application are used to provide a further understanding of the present application. Exemplary embodiments of the present application and descriptions thereof are used to explain the present application, and do not constitute an improper limitation to the present application.

FIG. 1 is a schematic diagram of microstructure distribution of an electrode array and curling shaping after film preparation of the present invention;

FIG. 2 is an appearance schematic diagram of an electrode array of the present invention; and

FIG. 3 is a sectional view of a film material of the present invention.

In the drawings:

1, electrode area;

2, lead area;

3, pin area;

4, support roller (or no reel);

5, stimulation electrode;

6, flexible biocompatible insulation material layer;

7, adhesion layer;

8, metal layer.

### DETAILED DESCRIPTION OF PREFERRED EMBODIMENTS

It should be noted that the following detailed description is exemplary and aims at further describing the present application. Unless otherwise specified, all technical and scientific terms used herein have the same meanings as commonly understood by those ordinary skilled in the art of the present application.

It should be noted that the terms used herein are only for describing specific embodiments, and are not intended to limit the exemplary embodiments according to the present application. As used herein, unless otherwise clearly specified in the context, the singular form is also intended to include the plural form. In addition, it should also be understood that the terms “comprising” and/or “including” used in the description indicate that there are features, steps, operations, devices, components, and/or combinations thereof.

As introduced in the background, the prior art has the problems that a cochlea implanted electrode is difficult in number increase of electrodes during the design and production, the electrodes are not firm enough and are easy to drop off sometimes, the individual electrode is difficult to design, and the operation requirements are high and are difficult to implement. In order to solve the above technical problems, the present invention proposes a flexible artificial auditory nerve stimulation electrode with a single-sided and overturned microstructure and a production method.

According to the position distribution rule of the cochlea auditory nerve endings, the present invention utilizes a flexible and biocompatible film material to design and produce the flexible electric stimulation electrode array by adopting the film as a substrate in steps of metal deposit, curling shaping, fixation and packaging, etc. The design and production method has the characteristics that the electrode is firm, the contacts can be densely distributed, the electrodes can be customized, the overall aperture of the electrode array is small, and the like, thereby meeting the individual and precise treatment.

### Embodiment 1

Specific steps adopted by the present invention are as follows:

Preparation Work:

A substrate is provided, such as a glass plate and a silicon wafer. Mask patterns of an electrode array are designed and include three portions, i.e. an electrode area 1, a lead area 2 and a pin area 3.

Step 1, the substrate is spin coated with a flexible biocompatible insulation material layer, such as PDMS, the rotation speed and time are controlled, and a film thickness is selected, such as 50-100 um, and then the substrate stands drying for solidification.

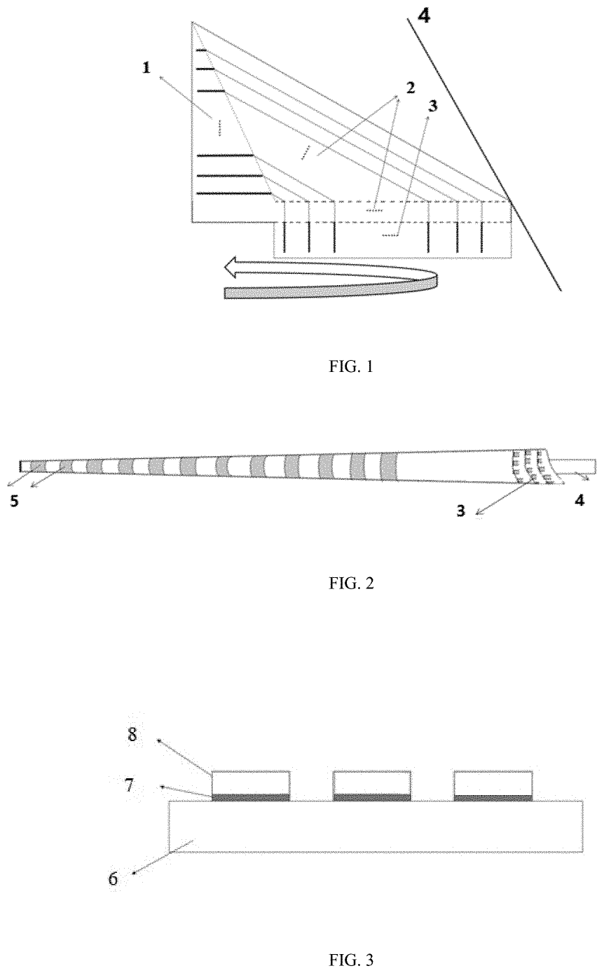

Step 2, the insulation material layer is spin coated with photoresist, and is then covered with a mask (as shown in FIG. 1, including the electrode area, the lead area and the pin area, but is not limited to the shape, and is cuttable), followed by exposure.

Step 3, developing is performed in a developing solution, followed by heating for a suitable time (such as 5 to 10 min) on a heating platform at a suitable temperature (such as 90° C.).

Step 4, by using electron-beam evaporation, an adhesion layer, such as titanium with a thickness of 3 to 20 nm, is deposited on the surface (or side) with the photoresist to improve the adhesion of the conductive metal.

Step 5, a conductive metal layer with good ductility, good conductivity and good biocompatibility, such as gold with an optional thickness (50 to 200 nm) is also deposited on the surface (or side) with the photoresist by using an electron-beam evaporation method as a conductive layer.

Step 6, the photoresist is washed, and then the processed and solidified flexible film with the metal conductive structure is stripped from the hard substrate.

Step 7, shaping: a long cylindrical or long conical filament is made into a support roller 4 (metal or nonmetal); the support roller is used as an axis, and the above electrode array film is curled at a given angle from a pin side (as shown in FIG. 1, on the right end) away from the electrode area in a manner that an insulation material side faces inside and the conductive metal layer faces outside. In this way, pins of all electrodes are exposed at a lower portion (as shown in FIG. 2, the pin area) of the support roller layer by layer. When the electrode array film is curled to the last circle (or last layer), all electrodes of the electrode area are almost vertically wound at an upper outermost layer of a long conical tube to approximately form an annular or U-shaped conductive electrode array. The lead area is layer-by-layer wrapped inside the long conical tube. Each lead is circumferentially separated by the insulation film material and insulated to one another.

Step 8, fixation and packaging: a suitable adhesive (such as liquid PDMS or glue) is smeared at the inner side of the tail end of the electrode area to bond the outermost curled edge, or the adhesive is smeared at the inner side of the film during the curling until the film is curled to the electrode area, followed by standing and fixation. Thereafter, the necessary electrode point-to-point electrical characteristic measurement and subsequent packaging of electronic circuits can be carried out.

Further, the film is curled into a corresponding shape, such as a cochlea needle shape, and a layered shape or a spherical crown shape at the auditory nucleus colliculi inferioris. Each electrode contact may be annular, U-shaped, etc. and depends on the curling and shaping.

In the needle-shaped electrode area of the cochlea electrode produced in this method, a diameter at the top cochleae surrounded by the electrodes may be 0.2 to 1.0 mm, and the diameter at the basis cochleae surrounded by the electrodes may be 1.0 to 5.0 mm.

Further preferably, a distance between the electrodes in adjacent electrode areas may be selected in a large range such as 50 um to 500 um according to the specific situation.

In order to improve the adhesion of the metal, an adhesion material layer may be added between the substrate insulation material layer and the metal conductive layer.

The bottom pin area (as shown in FIG. 1) is designed in a special shape, and the electrode pins may be exposed layer by layer during the curling of the film, thereby facilitating the subsequent test and connection.

Further preferably, during the shaping process, the long cylindrical or long conical filament is used as the support roller for curling, and each pin is exposed. The support roller is withdrawn outwards while being implanted when the electrode array is implanted into the cochlea, and finally the filament support roller is taken out.

The design and production method of the electrode has the characteristics that the electrodes are firm, the sensing electrode frequency resolution is high, the electrodes can be customized, the electrode needle is slender, etc., so that the individual and precise treatment for the hearing disability can be satisfied, and the electrode can be connected with various types of sound encoding processors.

Before the flexible insulation material layer is spin coated in step 1, the substrate is precoated with an anti-cohesion layer to facilitate the later film stripping.

The electrode area and the lead area are located in a same plane. During the shaping process, the filament support roller is used for curling, and the electrode pins are exposed. By curling, the electrode area is exposed at the upper portion of the long conical tube, and the pin area is exposed at the lower portion of the long conical tube (as shown in FIG. 2). The shape of each electrode contact may depend on a curling degree and may be customized according to actual requirements, for example, the electrode contact may be made in an annular shape, an u shape, a rectangular shape, etc. The bottom pin area of the plane electrode array (as shown in FIG. 1) may be designed in a special shape (such as a rectangle, a rhombus, a triangle, etc.), so that each pin may be exposed layer by layer during the curling, thereby facilitating the test and connection.

### Embodiment 2

The electrode produced from embodiment 1 includes a flexible biocompatible insulation material layer 6. An upper layer of the flexible biocompatible insulation material layer includes a conductive metal layer 8 and an adhesion layer 7. The conductive metal layer includes an electrode area 1, a lead area 2 and a pin area 3. A plurality of leads are etched in the lead area 2. A plurality of electrodes 5 are etched in the electrode area 1. One electrode is connected with one lead. The pin area 3 is provided with pins corresponding to the leads of the lead area 2 one by one. The electrodes 5 of the electrode area 1 are exposed outside and contact auditory nerve cell tissues to transfer a stimulation electric signal; and the lead area is wrapped inside the flexible biocompatible insulation material layer.

It can be seen from the above description that the above embodiments of the present application have the following technical effects:

1. The present invention adopts single-layer or two-layer PDMS, so that the thickness of the electrode material is reduced, and the electrode array with small aperture is easy to prepare, thereby facilitating the number increase of the electrodes, and guaranteeing the flexibility.

2. The conductive material adopted by the present invention is solid metal, the solid metal with good ductility and high conductivity may be made into the film, and the conductivity is relatively stable during the curling and use.

3. By using the solid metal, compared with the liquid metal, the present invention can avoid the possible secondary injury caused by the breaking leakage and is safer.

4. The electrode array produced by the present invention is easy for designing the annular or U-shaped electrode contacts, so that the conductive electrode is easy to face to and align at the cochlear axis, and during the implanting operation, the electrode is convenient to operate and easy to face to the auditory nerves.

5. Moreover, the present invention is good in conductivity, optimal in flexibility, safe (adopting the biocompatible material), precise (the size is small and the electrode contacts are exquisite), reliable (the electrode is not likely to drop off), convenient to shape (the flexible film material is curled by the support roller), and simple in preparation process. The present invention is small in volume and high in flexibility, and can reduce the physical injury during the cochlea implanting process.

The specific embodiments of the present invention are described above in combination with the accompanying drawings, but do not limit the protection scope of the present invention. Those skilled in the art shall understand that on the basis of the technical solutions of the present invention, various modifications or variations made by those skilled in the art without contributing creative efforts shall still fall within the protection scope of the present invention.

We claim:

1. A flexible single-sided conductive microstructure artificial cochlea electrode, comprising a flexible biocompatible insulation material layer, wherein an upper layer of the flexible biocompatible insulation material layer comprises a conductive metal layer and an adhesion layer; the conductive metal layer comprises an electrode area, a lead area and a pin area; a plurality of leads are etched in the lead area; a plurality of electrodes are etched in the electrode area; one electrode is connected with one lead; and the pin area is provided with pins corresponding to the leads of the lead area one by one.

2. The flexible single-sided conductive microstructure artificial cochlea electrode according to claim 1, wherein electrodes of the electrode area are exposed outside and contact auditory nerve cell tissues to transfer a stimulation electric signal; and the lead area is wrapped inside the flexible biocompatible insulation material layer.

3. The flexible single-sided conductive microstructure artificial cochlea electrode according to claim 1, wherein in a needle-shaped electrode area of the cochlea electrode, a diameter at the top cochleae surrounded by the electrodes may be 0.2 to 1.0 mm, and the diameter at the basis cochleae surrounded by the electrodes may be 1.0 to 5.0 mm.

4. A production method of the flexible single-sided conductive microstructure artificial cochlea electrode of claim 1, comprising the following steps:
step 1, providing a substrate, and coating the substrate with a flexible biocompatible insulation material layer;
step 2, spin coating the insulation material layer with photoresist, covering with a designed mask, and carrying out the exposure;
step 3, developing an exposed film in a developing solution, and heating at an appropriate temperature;
step 4, depositing or plating an adhesion layer at one side of the photoresist by using electron-beam evaporation;
step 5, depositing or plating a conductive metal layer at one side of the photoresist by using electron-beam evaporation;
step 6, washing the photoresist, or in order to protect a conductive structure, spin coating with another flexible insulation material (to properly avoid the electrode area), and solidifying, and stripping the solidified flexible biocompatible insulation material layer with the metal conductive structure from the hard substrate;
step 7, shaping; and
step 8, fixing and packaging.

5. The production method of the flexible single-sided conductive microstructure artificial cochlea electrode according to claim 4, wherein
a shaping method in step 7 is as follows:
a long cylindrical or long conical filament is used as a support roller; the prepared electrode array film is curled by adopting the support roller as an axis from a pin side away from the electrode area at a given angle or rolled from a film edge without a support reel, so that the pin area is exposed at the lower portion of the needle shape layer by layer; when the film is curled to a last circle, the electrodes of the electrode area are curled at the upper portion of the outermost layer of the support roller in an approximately vertical direction, thereby forming an approximately annular or U-shaped conductive electrode array; and the leads curled in the lead area are separated by the insulation film material and insulated to one another.

6. The production method of the flexible single-sided conductive microstructure artificial cochlea electrode according to claim 3, wherein
the process of step 8 is as follows: during the curling process, the film may be bonded while being curled; finally, a suitable adhesive is smeared at the inner side of the end of the electrode area until the outermost layer of the curled electrode area is adhered, or the film may be curled while smeared with the adhesive; after curling, standing and fixation are conducted; and then the necessary electrode point-to-point electrical characteristic measurement and subsequent package of the circuits may be carried out.

7. A production method of the flexible single-sided conductive microstructure artificial cochlea electrode, wherein the flexible biocompatible insulation material layer in step 1 may be polydimethylsiloxane (PDMS) or other flexible biocompatible insulation materials.
